SEQUENCE LISTING (21 sequences)

SEQ ID NO: 1 - protein, 8 aa, Artificial Sequence
  Synthetic
GFTFSSYA

SEQ ID NO: 2 - protein, 8 aa, Artificial Sequence
  Synthetic
IVGSGGST

SEQ ID NO: 3 - protein, 20 aa, Artificial Sequence
  Synthetic
AKSLIYGHYDILTGAYYFDY

SEQ ID NO: 4 - protein, 6 aa, Artificial Sequence
  Synthetic
QGIGNW

SEQ ID NO: 5 - protein, 3 aa, Artificial Sequence
  Synthetic
AAS

SEQ ID NO: 6 - protein, 8 aa, Artificial Sequence
  Synthetic
QQANSFPP

SEQ ID NO: 7 - protein, 127 aa, Artificial Sequence
  Synthetic
EVQLLESGGGLVQPGGSLRLSCAASGFTFSSYAMSWVRQAPGKGLEWVSAIVGSGGSTYYADSVKGRFIISRDNSKNTLYLQMNSLRAEDTAVYYCAKSLIYGHYDILTGAYYFDYWGQGTLVTVSS

SEQ ID NO: 8 - protein, 106 aa, Artificial Sequence
  Synthetic
DIQMTQSPSSVSASVGDRVTIPCRASQGIGNWLAWYQQKPGKAPKLLIYAASSLQSGVPSRFSGSGSGTDFTLTISSLQPEDFATYYCQQANSFPPFGQGTRLEIK

SEQ ID NO: 9 - DNA, 381 bp, Artificial Sequence
  Synthetic
gaggtgcagctgttggagtctgggggaggcttggtacagcctggggggtccctgagactctcctgtgcagcctctggattcacctttagcagctatgccatgagctgggttcgccaggctccagggaaggggctggagtgggtctcagctattgttggtagtggtggtagcacatactacgcagactccgtgaagggccggttcatcatctccagagacaattccaagaacactctgtatctgcaaatgaacagcctgagagccgaggacacggccgtttattactgtgcgaaatccctgatttatgggcattacgatattttgactggtgcttactactttgactactggggccagggaaccctggtcaccgtctcctca

SEQ ID NO: 10 - DNA, 318 bp, Artificial Sequence
  Synthetic
gacatccagatgacccagtctccatcttccgtgtctgcatctgtaggagacagagtcaccatcccttgtcgggcgagtcagggtattggcaactggttagcctggtatcagcagaaaccagggaaggcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctcccttcggccaagggacacgactggagattaaa

SEQ ID NO: 11 - protein, 223 aa, Artificial Sequence
  Amino acid sequence of the receptor binding domain of spike protein of SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 12 - protein, 330 aa, Homo sapiens [sequence omitted]

SEQ ID NO: 13 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 119 aa, Artificial Sequence
  Synthetic
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSS

SEQ ID NO: 16 - protein, 109 aa, Artificial Sequence
  Synthetic
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIK

SEQ ID NO: 17 - protein, 457 aa, Artificial Sequence
  Synthetic
EVQLLESGGGLVQPGGSLRLSCAASGFTFSSYAMSWVRQAPGKGLEWVSAIVGSGGSTYYADSVKGRFIISRDNSKNTLYLQMNSLRAEDTAVYYCAKSLIYGHYDILTGAYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 454 aa, Artificial Sequence
  Synthetic
EVQLLESGGGLVQPGGSLRLSCAASGFTFSSYAMSWVRQAPGKGLEWVSAIVGSGGSTYYADSVKGRFIISRDNSKNTLYLQMNSLRAEDTAVYYCAKSLIYGHYDILTGAYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 454 aa, Artificial Sequence
  Synthetic
EVQLLESGGGLVQPGGSLRLSCAASGFTFSSYAMSWVRQAPGKGLEWVSAIVGSGGSTYYADSVKGRFIISRDNSKNTLYLQMNSLRAEDTAVYYCAKSLIYGHYDILTGAYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 107 aa, Artificial Sequence [sequence omitted]

SEQ ID NO: 21 - protein, 213 aa, Artificial Sequence
  Synthetic
DIQMTQSPSSVSASVGDRVTIPCRASQGIGNWLAWYQQKPGKAPKLLIYAASSLQSGVPSRFSGSGSGTDFTLTISSLQPEDFATYYCQQANSFPPFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0399385 A1
(43) Pub. Date: Dec. 14, 2023

(54) NEUTRALIZING ANTIBODIES AGAINST SARS-COV-2

(71) Applicant: NANJING VAZYME BIOTECH CO., LTD., Nanjing, Jiangsu (CN)

(72) Inventors: Bai LU, Beijing (CN); Yang DOU, Beijing (CN); Lin CAO, Nanjing, Jiangsu (CN); Xiaoyu XU, Nanjing, Jiangsu (CN); Jianfeng PAN, Beijing (CN)

(21) Appl. No.: 18/245,155

(22) PCT Filed: Sep. 14, 2021
(86) PCT No.: PCT/CN2021/118164

§ 371 Date: Mar. 17, 2023

(30) Foreign Application Priority Data
Sep. 14, 2020 (WO) PCT/CN2020/115041

(51) Int. Cl. C07K 16/10; A61P 31/14; G01N 33/569; C12N 15/63

(52) U.S. Cl. CPC C07K 16/1003; A61P 31/14; G01N 33/56983; C12N 15/63; C07K 2317/565; C07K 2317/73; G01N 2800/26; C07K 2317/31

(57) ABSTRACT

Provided is a novel neutralizing antibody against spike protein of SARS-COV-2, and the antigen binding fragments thereof. Pharmaceutical composition and kits comprising the same, and the uses thereof are also provided.

### FIELD OF THE INVENTION

The present disclosure generally relates to novel neutralizing antibodies against SARS-COV-2.

### BACKGROUND

COVID-19 pandemics caused by a new member of coronavirus named Severe Acute Respiratory Syndrome-Corona Virus 2 (SARS-CoV-2) has rapidly spread around the world. SARS-CoV-2 encodes a spike (S) glycoprotein on the surface, which has two functional subunits S1 and S2. The S1 subunit contains receptor-binding domain (RBD) which binds to human angiotensin converting enzyme 2 (ACE2) receptor directly. The spike glycoprotein of SARS-CoV-2 mediates the viral entry into human host cells. To date, no targeted drugs are available for COVID-19 disease.

Therapeutic monoclonal antibody (mAb) had been approved for treatment of many diseases. Neutralizing antibodies therapies have shown to be effective in treating virus infections. Antibody mAb 114 isolated from a human survivor of 1995 Kikwit Ebola virus disease showed a strong neutralizing activity against Ebola virus. Clinical study found that mAb 114 significantly reduced mortality of patients suffering Ebola disease.

However, effective neutralizing antibodies for SARS-COV-2 are still lacking. Therefore, there is a need for neutralizing antibodies with potent neutralizing effects on SARS-COV-2.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one respect, the present disclosure provides an isolated antibody or an antigen-binding fragment thereof capable of specifically binding to spike protein (e.g. S1) of SARS-CoV-2, comprising a heavy chain CDR 1 (HCDR1), HCDR2 and HCDR3 and/or a light chain CDR1 (LCDR1), LCDR2 and LCDR3, wherein: the HCDR1, the HCDR2, and the HCDR3 comprise amino acid sequences of SEQ ID NO: 1, SEQ ID NO: 2, and SEQ ID NO: 3, respectively, and the LCDR1, the LCDR2, and the LCDR3 comprise amino acid sequences of SEQ ID NO: 4, SEQ ID NO: 5, and SEQ ID NO: 6, respectively.

In some embodiments, the antibody or an antigen-binding fragment thereof provided herein further comprises a heavy chain variable region (VH) comprising an amino acid sequence of SEQ ID NO: 7, or a sequence having at least 80% sequence identity thereof.

In some embodiments, the antibody or an antigen-binding fragment thereof provided herein, further comprising a light chain variable region (VL) comprising an amino acid sequence of SEQ ID NO: 8, or a sequence having at least 80% sequence identity thereof.

In some embodiments, the antibody or antigen-binding fragment provided herein comprises: a VH comprising an amino acid sequence of SEQ ID NO: 7 or a sequence having at least 80% sequence identity thereof, and a VL comprising an amino acid sequence of SEQ ID NO: 8 or a sequence having at least 80% sequence identity thereof.

In some embodiments, the antibody or antigen-binding fragment thereof provided herein binds to receptor binding domain (RBD) of spike protein of SARS-CoV-2, for example, RBD of S1 of SARS-CoV-2.

In some embodiments, the antibody or antigen-binding fragment thereof provided herein further comprises one or more amino acid residue mutations yet retains specific binding to spike protein (e.g. S1) of SARS-CoV-2. In some embodiments, the one or more amino acid residue mutations improve drug-like properties such as stability, pharmacokinetic/pharmacodynamic properties, yield of production, and reduced toxicity, and so on.

In some embodiments, at least one of the mutations is in one or more of the CDR sequences, and/or in one or more of the VH or VL sequences but not in any of the CDR sequences.

In some embodiments, the antibody or antigen-binding fragment thereof provided herein further comprises an immunoglobulin constant region, optionally a constant region of human Ig (e.g. human IgG1, IgG2, IgG3, IgG4, IgA1, IgA2 or IgM), or optionally a constant region of human IgG.

In some embodiments, the constant region comprises a constant region of human IgG1 or IgG4.

In some embodiments, the heavy chain constant region of human IgG1 comprises SEQ ID NO: 12, or a sequence having at least 80% sequence identity thereof. In some embodiments, the heavy chain constant region of human IgG4 comprises SEQ ID NO: 13, or a sequence having at least 80% sequence identity thereof.

In some embodiments, the Fc region comprises one or more amino acid residue mutations conferring increased or reduced complement dependent cytotoxicity (CDC) or complement dependent cytotoxicity (ADCC) relative to wild-type constant region.

In some embodiments, the Fc region does not contribute to antibody dependent enhancement (ADE) of SARS-CoV-2 infection. In some embodiments, the Fc region comprise one or more mutations that reduce the binding of the antibody to Fc receptor. In certain embodiments, the antibodies or antigen-binding fragments thereof provided herein lack an Fc region and hence do not bind to Fc receptor.

In some embodiments, the antibody or an antigen-binding fragment thereof provided herein is fully human antibody, chimeric antibody, monoclonal antibody, a bispecific antibody, a multi-specific antibody, recombinant antibody, labeled antibody, bivalent antibody, anti-idiotypic antibody or a fusion protein.

In some embodiments, the antibody or antigen-binding fragment thereof provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In some embodiments, the antibody or antigen-binding fragment thereof provided herein is bispecific.

In some embodiments, the bispecific antibody or antigen-binding fragment thereof provided herein is capable of specifically binding to distinct epitopes on spike protein of SARS-CoV-2 or distinct antigens of SARS-CoV-2. In some embodiments, the bispecific antibody or antigen-binding fragment thereof provided herein is capable of specifically binding to distinct epitopes on S1 subunit of spike protein of SARS-CoV-2 or distinct subunits of spike protein of SARS-CoV-2.

In some embodiments, the antibody or antigen-binding fragment thereof provided herein is linked to one or more conjugate moieties.

In another aspect, the prevent disclosure provides an antibody or an antigen-binding fragment thereof, which competes for binding to RBD of spike protein of SARS-CoV-2 with the antibody or antigen-binding fragment thereof comprising the CDR sequences provided herein.

In another aspect, the present disclosure provides a composition comprising a combination of one or more antibodies or antigen-binding fragments. In certain embodiments, the combination comprises antibodies or antigen-binding fragment thereof binding to distinct epitopes on spike protein of the SARS-CoV-2. In certain embodiments, the combination comprises antibodies or antigen-binding fragment thereof binding to distinct subunits of spike protein of the SARS-CoV-2. In certain embodiments, the combination comprises two or more antibodies which specifically bind to SARS-CoV-2 in a non-competing manner.

In another aspect, the present disclosure provides a pharmaceutical composition comprising one or more of the antibodies or antigen-binding fragments thereof, and a pharmaceutically acceptable carrier.

In certain embodiments, the pharmaceutical composition comprises a combination of two or more of the antibodies or antigen-binding fragments thereof. In certain embodiments, the two or more of the antibodies or antigen-binding fragments thereof in the combination bind to distinct epitopes on spike protein of the SARS-CoV-2. In certain embodiments, the two or more of the antibodies or antigen-binding fragments thereof in the combination specifically bind to SARS-CoV-2 in a non-competing manner.

In certain embodiments, the pharmaceutical composition further comprises an additional antibody capable of neutralizing SARS-CoV-2.

In certain embodiments, the additional antibody is capable of binding to SARS-CoV-2 at an epitope or antigen distinct from that/those bound by the antibodies or antigen-binding fragments.

In certain embodiments, the pharmaceutical composition further comprises an additional antibody capable of binding to RBD of spike protein of the SARS-CoV-2 at an epitope different from that/those bound by Antibody 5-10.

In embodiments, the additional antibody is capable of binding to non-RBD region of spike protein of the SARS-CoV-2.

In certain embodiments, the pharmaceutical composition provided herein comprises a cocktail of SARS-CoV-2 neutralizing antibodies that binds to at least two (at least 3, at least 4, etc.) distinct epitopes on a SARS-CoV-2 serotype or two or more SARS-CoV-2 serotypes.

In another aspect, the present disclosure provides an isolated polynucleotide encoding the antibody or an antigen-binding fragment thereof of the present disclosure.

In another aspect, the present disclosure provides a vector comprising the isolated polynucleotide provided herein, optionally the vector is an expression vector.

In another aspect, the present disclosure provides a host cell comprising the vector of the present disclosure.

In another aspect, the present disclosure provides a method of expressing the antibody or antigen-binding fragment thereof of the present disclosure, comprising culturing the host cell of the present disclosure under the condition at which the vector provided herein is expressed.

In another aspect, the present disclosure provides a composition comprising a first mRNA polynucleotide encoding heavy chain or an antigen-binding fragment thereof of the antibody of the present disclosure, and a second mRNA polynucleotide encoding light chain or a fragment thereof of the antibody of the present disclosure.

In certain embodiments, the composition provided herein further comprises a pharmaceutically acceptable carrier.

In another aspect, the present disclosure provides a method of producing the antibody of the present disclosure, which comprising administering the composition provided herein to a cell, wherein the first mRNA polynucleotide and the second mRNA polynucleotide are expressed in the cell, thereby producing the antibody.

In another aspect, the present disclosure provides a method of delivering the antibody of the present disclosure, which comprising administering the composition provided herein to a subject in need thereof, wherein the first mRNA polynucleotide and the second mRNA polynucleotide are expressed in the cell, thereby producing the antibody.

In another aspect, the present disclosure provides a method of ameliorating, treating or preventing SARS-CoV-2 infection in a subject, comprising administering to the subject an effective amount of the antibody or antigen-binding fragment thereof, the pharmaceutical composition, or the composition provided herein of the present disclosure.

In certain embodiments, the subject is human or a non-human animal.

In certain embodiments, the subject has been identified as having SARS-CoV-2 infection, or is suspected of having SARS-CoV-2 infection, or is at risk of exposure to SARS-CoV-2.

In certain embodiments, the administration is via oral, nasal, intravenous, subcutaneous, sublingual, or intramuscular administration.

In certain embodiments, the method provided herein further comprises administering an effective amount of a second therapeutic agent.

In certain embodiments, the second therapeutic agent is selected from a second SARS-CoV-2 neutralizing antibody, an antiviral agent such as RNA dependent RNA polymerase inhibitor, a nucleoside analog, antiviral cytokines (such as interferons), or immunostimulatory agents.

In another aspect, the present disclosure provides a kit comprising an antibody of the present disclosure, and a second therapeutic agent.

In another aspect, the present disclosure provides a method of neutralizing SARS-CoV-2 in a subject, comprising administering the antibody, antigen-binding fragment thereof, or the composition provided herein of the present disclosure.

In another aspect, the present disclosure provides a method for preventing or reducing transmission of SARS-CoV-2 by a SARS-CoV-2 infected subject, comprising administering to the SARS-CoV-2 infected subject an effective amount of the antibody or antigen-binding fragment thereof, and/or the pharmaceutical composition, and/or the composition provided herein of the present disclosure.

In another aspect, the present disclosure provides a method of preventing or reducing ameliorating or treating a subject infected with SARS-CoV-2, or inhibiting transmission of SARS-CoV-2 by the subject infected with SARS-CoV-2, comprising administering to the subject an effective amount of the antibody or antigen-binding fragment thereof, and/or the pharmaceutical composition, and/or the composition provided herein of the present disclosure.

In another aspect, the present disclosure provides a method of reducing viral load in a SARS-CoV-2 infected subject, comprising administering to the subject an effective amount of the antibody or antigen-binding fragment thereof, and/or the pharmaceutical composition, and/or the composition provided herein of the present disclosure.

In another aspect, the present disclosure provides a method of diagnosing SARS-CoV-2 infection in a subject, comprising: a) contacting a sample obtained from the subject with the antibody or antigen-binding fragment thereof of the present disclosure; b) determining presence or amount of SARS-CoV-2 in the sample; and c) correlating the presence or the amount of SARS-CoV-2 to existence or status of the SARS-CoV-2 infection in the subject.

In another aspect, the present disclosure provides use of the antibody or antigen-binding fragment thereof, and/or the composition provided herein of the present disclosure in the manufacture of a medicament for treating or preventing SARS-CoV-2 infection in a subject; or for preventing, inhibiting progression of, and/or delaying the onset of SARS-CoV-2 infection or a SARS-CoV-2-associated condition in a subject; or for preventing or reducing transmission of SARS-CoV-2 by a SARS-CoV-2 infected subject; or for reducing viral load in a SARS-CoV-2 infected subject.

In another aspect, the present disclosure provides use of the antibody or antigen-binding fragment thereof, and/or the composition provided herein of the present disclosure in the manufacture of a diagnostic reagent for diagnosing SARS-CoV-2 infection.

In another aspect, the present disclosure provides a kit comprising the antibody or antigen-binding fragment thereof of the present disclosure, useful in detecting SARS-CoV-2 presence.

### BRIEF DESCRIPTION OF THE DRAWINGS

FIG. 1 shows the plot between the log concentrations of Antibody 5-10 and OD450 as measured by enzyme-linked immunosorbent assay (ELISA), indicating the binding of Antibody 5-10 to RBD of spike protein of SARS-CoV-2.

FIG. 2 shows binding kinetics of Antibody 5-10 using biolayer interferometry.

FIG. 3 shows capability of Antibody 5-10 to block interaction between ACE2 and RBD of SARS-CoV-2, as measured using Homogeneous Time-Resolved Fluorescence (HTRF) technology.

FIG. 4 shows SARS-CoV-2 S pseudotyped virus neutralization assay results for Antibody 5-10 as measured by luciferase reporter.

FIG. 5 shows capability of Antibody 5-10 to neutralize wild-type SARS-CoV-2 pseudovirus.

FIG. 6 shows comparison of viral load in mouse lungs when Antibody 5-10 was used for prophylaxis and treatment.

FIG. 7 shows the amino acid sequences of IgG1, IgG4, IgG4 mutant, RBD, JS016 VH, JS016 VL, heavy chain of Antibody 5-10 and light chain of Antibody 5-10.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are large immunoglobulin A (IgA), IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragments include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond. The heavy chain fragment of the Fab is known as “Fd”.

“Fab′” refers to a Fab fragment that includes a portion of the hinge region.

“F(ab′)₂” refers to a dimer of Fab′.

“Fc” with regard to an antibody (e.g. of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)).

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a V_{H} domain connected to a V_{L} domain in the same polypeptide chain (V_{H}-V_{L} or V_{L}-V_{H}) (see, e.g. Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

In certain embodiments, an “scFv dimer” is a bivalent diabody or bispecific scFv (BsFv) comprising V_{H}-V_{L} (linked by a peptide linker) dimerized with another VH-VL moiety such that VH'S of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g. a long flexible linker) and bound to two V_{L} moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In some embodiments, the non-human animal is a mammal, for example, a mouse, a rat, a rabbit, a goat, a sheep, a guinea pig, or a hamster.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (k_{off}/kₒₙ) when the binding between the antigen and antigen-binding molecule reaches equilibrium. K_{D} may be determined by using any conventional method known in the art, including but are not limited to surface plasmon resonance method, Octet method, microscale thermophoresis method, HPLC-MS method and FACS assay method. A K_{D} value of ≤10⁻⁶ M (e.g. ≤5×10⁻⁷ M, ≤2×10⁻⁷ M, ≤10⁻⁷ M, ≤5×10⁻⁸ M, ≤2×10⁻⁸ M, ≤10⁻⁸M, ≤5×10⁻⁹ M, ≤4×10⁻⁹M, ≤3×10⁻⁹M, ≤2×10⁻⁹ M, or ≤10⁻⁹ M) can indicate specific binding between an antibody or antigen binding fragments thereof and spike protein of SARS-CoV-2 (e.g. RBD of spike protein of SARS-CoV-2).

“Receptor binding domain” or “RBD” of spike protein of SARS-CoV-2 refers to a domain of a spike (S) glycoprotein (in particular, the S1 subunit thereof) of a SARS-CoV-2 virus, which domain is capable of binding to or engaging with a host cell receptor angiotensin-converting enzyme 2 (ACE2). After binding of RBD to ACE2, the S2 subunit of the S glycoprotein mediates fusion between the viral and host cell membrane, to facilitate the entry of the virus particle into the host cell. The RBD region from the full-length amino acid sequence of the S glycoprotein of SARS-CoV-2 can be identified using methods or modified version thereof as described in Tai, W., He, L., Zhang, X. et al., Cell Mol Immunol 17, 613-620 (2020); Tai, W. et al., J. Virol. 91, 01651-16 (2017); Ma, C. et al., Vaccine 32, 6170-6176 (2014). An illustrative example of amino acid sequence of the “RBD” is set forth in SEQ ID NO: 11, but a skilled person would understand that RBD sequences can mutate with the SARS-CoV-2 virus and therefore can have a variety of variants and mutants, which are intended to be encompassed by the term in the present disclosure.

The ability to “compete for binding to RBD of spike protein of SARS-CoV-2” as used herein refers to the ability of a first antibody or antigen-binding fragment to inhibit the binding interaction between RBD of spike protein of SARS-CoV-2 and a second antibody to any detectable degree. In certain embodiments, an antibody or antigen-binding fragment that compete for binding to RBD of spike protein of SARS-CoV-2 inhibits the binding interaction between RBD of spike protein of SARS-CoV-2 and a second antibody binding to RBD of spike protein of SARS-CoV-2 by at least 80%, 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e. including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

“Antibody dependent enhancement” or “ADE” as used herein refers to a situation where a subject having two sequential exposures to a virus (e.g. SARS-CoV-2) of different serotypes, could experience more severe infection in the second exposure than in the first exposure, for example having more severe symptoms, or more likely to have disease progression. More details are found, for example, in Balsitis et al., PLoS Pathog 6(2): e1000790. The mechanism for ADE could be that, an anti-viral antibody binds simultaneously to the virus and to a host cell (believed to be mediated via the Fcγ receptor), thereby increasing infectivity.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g. expression vectors) containing the nucleic acid sequence provided herein encoding the antibody or an antigen-binding fragment thereof, at least one promoter (e.g. SV40, CMV, EF-1α) operably linked to the nucleic acid sequence, and at least one selection marker.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

The term “prevent” or “preventing” as used herein includes slowing the onset of a disease, reducing the risk of developing a disease, suppressing or delaying the manifestation or development of symptoms associated with a disease, reducing the severity of a subsequent contraction or development of a disease, ameliorating a related symptom, and inducing immunity to protect against a disease,

The term “neutralizing” with respect to an antibody means that the antibody is capable of disrupting a formed viral particle or inhibiting formation of a viral particle or prevention of binding or infection of susceptible cells by a viral particle.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a RBD of spike protein of SARS-CoV-2 related disease, or refer to identification of a subject with a RBD of spike protein of SARS-CoV-2 related disease who may benefit from a particular treatment regimen.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a pharyngeal swab, a blood, sputum, feces, urine, or nasal sample. In some embodiments, the biological sample is Bronchoalveolar lavage fluid and fibrobronchoscope brush biopsy.

The term “antibody against spike protein of SARS-CoV-2” refers to an antibody that is capable of specific binding to spike protein of SARS-CoV-2.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Anti-SARS-CoV-2 Antibodies

The present disclosure provides neutralizing antibodies against SARS-CoV-2 and antigen-binding fragments of such neutralizing antibodies. The neutralizing antibodies against SARS-CoV-2 and antigen-binding fragments thereof provided herein are capable of specifically binding to spike protein of SARS-CoV-2, in particular, specifically binding to RBD of spike protein of SARS-CoV-2. Combinations of the neutralizing antibodies are also encompassed by the present disclosure.

In certain embodiments, the antibodies and the antigen-binding fragments thereof provided herein specifically bind to spike protein of SARS-CoV-2 at a K_{D} value of no more than 10⁻⁸ M, no more than 8×10⁻⁹ M, no more than 5×10⁻⁹ M, no more than 4×10⁻⁹ M, no more than 3×10⁻⁹ M, no more than 2×10⁻⁹ M, no more than 1×10⁻⁹ M, no more than 8×10⁻¹⁰ M, no more than 6×10⁻¹⁰ M, no more than 4×10⁻¹⁰ M, no more than 2×10⁻¹⁰ M, no more than 10⁻¹⁰ M, no more than 9×10⁻¹¹ M, no more than 8×10⁻¹¹ M, no more than 7×10⁻¹¹ M, no more than 6×10⁻¹¹ M, no more than 5×10⁻¹¹ M, no more than 4×10⁻¹¹ M, or no more than 3×10⁻¹¹ M using biolayer interferometry. In certain embodiments, the K_{D} value is measured by the method as described in Example 2 of the present disclosure.

In certain embodiments, the antibodies and antigen-binding fragments thereof provided herein exhibits competitive RBD binding property that effectively blocks the binding of RBD of spike protein of SARS-CoV-2 to ACE2 on surface of a host cell to block entry of SARS-CoV-2 into the host cell. The SARS-CoV-2 blocking effect or neutralizing effect of the antibodies and antigen-binding fragments thereof provided herein can be measured using pseudovirus blocking methods as described in, for example, Example 2 of the present disclosure. In certain embodiments, the blocking effect or neutralizing effect on SARS-CoV-2 pseudovirus of the antibodies and antigen-binding fragments thereof provided herein can be expressed in IC50, which indicates the concentration of the antibodies and antigen-binding fragments thereof provided herein to decrease 50% of the binding of SARS-CoV-2 pseudovirus RBD to ACE2 is decreased by 50% in presence of the antibodies and antigen-binding fragments thereof of the present disclosure. In certain embodiments, the pseudovirus blocking IC50 of the antibodies and antigen-binding fragments thereof provided herein is in a range from 0.003 μg/mL to 5 μg/mL, from 0.003 μg/mL to 0.9 μg/mL, from 0.003 μg/mL to 0.1 μg/mL, from 0.003 μg/mL to 0.09 μg/mL, from 0.003 μg/mL to 0.05 μg/mL, from 0.003 μg/mL to 0.04 μg/mL, from 0.003 μg/mL to 0.03 μg/mL, from 0.003 μg/mL to 0.02 μg/mL, or from 0.003 μg/mL to 0.01 μg/mL. In certain embodiments, the pseudovirus blocking IC50 of the antibodies and antigen-binding fragments thereof provided herein is less than 1 μg/mL, less than 0.5 μg/mL, less than 0.05, less than 0.04 μg/mL, or less than 0.01 μg/mL.

The SARS-CoV-2 blocking effect or neutralizing effect of the antibodies and antigen-binding fragments thereof provided herein can also be measured using live virus blocking methods as described in, for example, Example 2 of the present disclosure.

Illustrative Neutralizing Antibodies Against Spike Protein of SARS-CoV-2

In certain embodiments, the present disclosure provides neutralizing antibodies against spike protein of SARS-CoV-2 and antigen-binding fragments thereof comprising one or more (e.g. 1, 2, 3, 4, 5, or 6) CDRs comprising the sequences selected from the group consisting of GFTFSSYA (SEQ ID NO: 1), IVGSGGST (SEQ ID NO: 2), AKSLIYGHYDILTGAYYFDY (SEQ ID NO: 3), QGIGNW (SEQ ID NO: 4), AAS (SEQ ID NO: 5), and QQANSFPP (SEQ ID NO: 6).

Antibody “5-10” as used herein refers to a monoclonal antibody having a heavy chain variable region having the sequence of SEQ ID NO: 7, and a light chain variable region having the sequence of SEQ ID NO: 8.

In certain embodiments, the present disclosure provides neutralizing antibodies against spike protein of SARS-CoV-2 and antigen-binding fragments thereof comprising one or more (e.g. 1, 2, 3, 4, 5, or 6) CDR sequences of Antibody 5-10.

In certain embodiments, the present disclosure provides neutralizing antibodies against spike protein of SARS-CoV-2 and antigen-binding fragments thereof comprising a HCDR1 comprising the sequence of SEQ ID NO: 1, a HCDR2 comprising the sequence of SEQ ID NO: 2, a HCDR3 comprising the sequence of SEQ ID NO: 3, and/or a LCDR1 comprising the sequence of SEQ ID NO: 4, a LCDR2 comprising the sequence of SEQ ID NO: 5, and a LCDR3 comprising the sequence of SEQ ID NO: 6.

Table 1 below shows the CDR amino acid sequences of Antibody 5-10. The CDR boundaries for 5-10 were defined or identified by the convention of IMGT. Table 2 below shows the heavy chain and light chain variable region amino acid sequences of Antibody 5-10. Table 3 below shows the heavy chain and light chain variable region nucleic acid sequences of Antibody 5-10.

TABLE 1
CDR amino acid sequences of Antibody 5-10.

| | | CDR1 | CDR2 | CDR3 |
|---|---|---|---|---|
| 5-10 | HCDR | SEQ ID | SEQ ID | SEQ ID |
| | | NO: 1 | NO: 2 | NO: 3 |
| | | GFTFSSYA | IVGSGGST | AKSLIYG |
| | | | | HYDILTG |
| | | | | AYYFDY |
| | LCDR | SEQ ID | SEQ ID | SEQ ID |
| | | NO: 4 | NO: 5 | NO: 6 |
| | | QGIGNW | AAS | QQANSFPP |

TABLE 2
Variable region amino acid sequences
of Antibody 5-10.

| | VH | VL |
|---|---|---|
| 5-10 | SEQ ID NO: 7 | SEQ ID NO: 8 |
| | EVQLLESGGGLVQPG | DIQMTQSPSSVSASV |
| | GSLRLSCAASGFTFS | GDRVTIPCRASQGIG |
| | SYAMSWVRQAPGKGL | NWLAWYQQKPGKAPK |
| | EWVSAIVGSGGSTYY | LLIYAASSLQSGVPS |
| | ADSVKGRFIISRDNS | RFSGSGSGTDFTLTI |
| | KNTLYLQMNSLRAED | SSLQPEDFATYYCQQ |
| | TAVYYCAKSLIYGHY | ANSFPPFGQGTRLEI |
| | DILTGAYYFDYWGQG | K |
| | TLVTVSS | |

TABLE 3
Variable region nucleic acid sequences
of Antibody 5-10.

| | VH | VL |
|---|---|---|
| 5-10 | SEQ ID NO: 9 | SEQ ID NO: 10 |
| | gaGGTGCAGCTGTTG | gaCATCCAGATGACC |
| | GAGTCTGGGGGAGGC | CAGTCTCCATCTTCC |
| | TTGGTACAGCCTGGG | GTGTCTGCATCTGTA |
| | GGGTCCCTGAGACTC | GGAGACAGAGTCACC |
| | TCCTGTGCAGCCTCT | ATCCCTTGTCGGGCG |
| | GGATTCACCTTTAGC | AGTCAGGGTATTGGC |
| | AGCTATGCCATGAGC | AACTGGTTAGCCTGG |
| | TGGGTTCGCCAGGCT | TATCAGCAGAAACCA |
| | CCAGGGAAGGGGCTG | GGGAAGGCCCCTAAG |
| | GAGTGGGTCTCAGCT | CTCCTGATCTATGCT |
| | ATTGTTGGTAGTGGT | GCATCCAGTTTGCAA |
| | GGTAGCACATACTAC | AGTGGGGTCCCATCA |
| | GCAGACTCCGTGAAG | AGGTTCAGCGGCAGT |
| | GGCCGGTTCATCATC | GGATCTGGGACAGAT |
| | TCCAGAGACAATTCC | TTCACTCTCACCATC |
| | AAGAACACTCTGTAT | AGCAGCCTGCAGCCT |
| | CTGCAAATGAACAGC | GAAGATTTTGCAACT |
| | CTGAGAGCCGAGGAC | TACTATTGTCAACAG |
| | ACGGCCGTTTATTAC | GCTAACAGTTTCCCT |
| | TGTGCGAAATCCCTG | CCCTTCGGCCAAGGG |
| | ATTTATGGGCATTAC | ACACGACTGGAGATT |
| | GATATTTTGACTGGT | AAA |
| | GCTTACTACTTTGAC | |
| | TACTGGGGCCAGGGA | |
| | ACCCTGGTCACCGTC | |
| | TCCTCA | |

CDRs are known to be responsible for antigen binding. However, it has been found that not all of the 6 CDRs are indispensable or unchangeable. In other words, it is possible to replace or change or modify one or more CDRs in neutralizing Antibody 5-10, yet substantially retain the specific binding affinity to spike protein of SARS-CoV-2.

Antibody 5-10 and antigen-binding fragments thereof provided herein can comprise suitable framework region (FR) sequences from any species, such as mouse, human, rat, or rabbit, as long as the antibodies and antigen-binding fragments thereof can specifically bind to spike protein of SARS-CoV-2. In certain embodiments, the CDR sequences provided in Table 1 above are obtained from human antibodies. In certain embodiments, the FR sequence is derived from human.

In certain embodiments, the antibodies and antigen-binding fragments thereof provided herein are fully human. The term “fully human” antibody as used herein, with reference to an antibody or antigen-binding domain, means that the antibody or the antigen-binding domain has or consists of amino acid sequence(s) corresponding to that of an antibody produced by a human or a human immune cell, or derived from a non-human source such as a transgenic non-human animal that utilizes human antibody repertoires or other human antibody-encoding sequences. In certain embodiments, a fully human antibody does not comprise amino acid residues (in particular antigen-binding residues) derived from a non-human antibody. A fully human antibody may contain one or more mutations (e.g. substitutions, insertions or deletions) relative to the corresponding germline sequences. For example, one or more amino acid residues can be mutated to the corresponding residue(s) of the germline sequence from which the antibody was derived (i.e. back-mutation), or to the corresponding residue(s) of another human germline sequence, or to a conservative amino acid substitution of the corresponding germline residue(s). In some embodiments, back mutations can be introduced to one or more framework regions or CDR regions. Such back-mutations are desirable in some embodiments to reduce immunogenicity. In some other embodiments, an amino acid residue in one human germline sequence may be substituted to the corresponding amino acid residue in a second human germline sequence which is different from the germline sequence from which the antibody is originally derived.

The one or more mutations of the fully human antibody can comprise may be present in CDR regions or non-CDR regions (e.g., FR regions) of heavy and/or light chains, which endows altered (increased or decreased) properties of the full human antibody, including but not limited to, immunogenicity, binding affinity, binding specificity, antagonistic or agonistic biological properties. In some embodiments, the one or more amino acid residue mutations improve drug-like properties such as stability, pharmacokinetic/pharmacodynamic properties, yield of production, and reduced toxicity, and so on. This can be achieved by various mutagenesis technologies known in the art, such as site-directed mutagenesis, PCR mutagenesis, insertional mutagenesis, signature tagged mutagenesis (STM), transposon mutagenesis, or sequence saturation mutagenesis (SeSaM) (Hsu P D, Lander E S, Zhang F (June 2014). Cell. 157 (6): 1262-78; Carlson C M, Largaespada D A (July 2005). Nat. Rev. Genet. 6 (7): 568-80; Saenz H L, Dehio C (October 2005). Curr. Opin. Microbiol. 8 (5): 612-9; Seifert, H S; Chen, E Y; So, M; Heffron, F (1986 Feb. 1). Proceedings of the National Academy of Sciences of the United States of America. 83 (3): 735-739; Mundhada, H.; Marienhagen, J.; Scacioc, A.; Schenk, A.; Roccatano, D.; Schwaneberg, U. (2011). ChemBioChem. 12 (10): 1595-1601.).

In some embodiments, the FR regions derived from human may comprise the same amino acid sequence as the human immunoglobulin from which it is derived. In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity.

In some embodiments, the antibodies and antigen-binding fragments thereof provided herein comprise all or a portion of the heavy chain variable domain and/or all or a portion of the light chain variable domain. In one embodiment, the antibodies and antigen-binding fragments thereof provided herein is a single domain antibody which consists of all or a portion of the heavy chain variable domain provided herein. More information of such a single domain antibody is available in the art (see, e.g. U.S. Pat. No. 6,248,516).

In certain embodiments, the antibodies and antigen-binding fragments thereof provided herein further comprise an immunoglobulin (Ig) constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the antibodies and antigen-binding fragments thereof provided herein comprises heavy chain constant regions of human IgG1, IgG2, IgG3, IgG4, IgA1, IgA2 or IgM. In certain embodiments, the antibodies and antigen-binding fragments thereof provided herein comprises heavy chain constant regions of human IgG1. In certain embodiments, the antibodies and antigen-binding fragments thereof provided herein comprises heavy chain constant regions of human IgG4. In certain embodiments, the light chain constant region comprises Cκ or Cλ. The constant region of the antibodies and antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the antibodies and antigen-binding fragments thereof provided herein comprises an Fc region of IgG1, or IgG3 isotype, which could induce ADCC or CDC; or alternatively, a constant region of IgG4 or IgG2 isotype, which has reduced or depleted effector function. In some embodiments, the Fc region is derived from human IgG1 with reduced effector functions. In certain embodiments, the antibodies and antigen-binding fragments thereof provided herein comprise a wild type human IgG1 Fc region or other wild type human IgG1 alleles. In some embodiments, the heavy chain constant region derived from human IgG1 comprises an amino acid sequence having at least 80%, at least 85%, at least 90%, or at least 95% sequence identity of SEQ ID NO: 12. In some embodiments, the heavy chain constant region derived from human IgG1 comprises an amino acid sequence of SEQ ID NO: 12. In certain embodiments, the antibodies and antigen-binding fragments thereof provided herein comprise a human IgG1 Fc region comprising one or more mutations, which can confer increased CDC or ADCC relative to wild-type constant region.

In certain embodiments, the antibodies and antigen-binding fragments thereof provided herein further comprises a light chain constant region comprising an amino acid sequence of SEQ ID NO: 20.

In certain embodiments, the antibodies and antigen-binding fragments thereof provided herein comprises a heavy chain comprising an amino acid sequence of SEQ ID NO: 17, 18, or 19. In certain embodiments, the antibodies and antigen-binding fragments thereof provided herein comprises a light chain comprising an amino acid sequence of SEQ ID NO: 21.

In some embodiments, the heavy chain constant region derived from human IgG4 comprises an amino acid sequence having at least 80%, at least 85%, at least 90%, or at least 95% sequence identity of SEQ ID NO: 13. In some embodiments, the heavy chain constant region derived from human IgG4 comprises an amino acid sequence of SEQ ID NO: 13. In certain embodiments, the antibodies and antigen-binding fragments thereof provided herein comprise a human IgG4 Fc region comprising a S228P mutation, a F234A mutation, and/or a L235A mutation (see, e.g. SEQ ID NO: 14), which confers decreased CDC or ADCC relative to wild-type constant region.

In certain embodiments, the antibodies or the antigen-binding fragments thereof provided herein have a specific binding affinity to spike protein of SARS-CoV-2 which is sufficient to provide for diagnostic and/or therapeutic use.

In certain embodiments, Antibody 5-10 or the antigen-binding fragments thereof provided herein bind to receptor binding domain (RBD) of spike protein of SARS-CoV-2.

The antibodies or antigen-binding fragments thereof provided herein can be a monoclonal antibody, a polyclonal antibody, a humanized antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides a neutralizing antibody or antigen-binding fragment thereof, which competes for binding to spike protein of SARS-CoV-2 with the antibody or antigen-binding fragment thereof provided herein. In certain embodiments, the present disclosure provides a neutralizing antibody or antigen-binding fragment thereof, which competes for binding to spike protein of SARS-CoV-2 with an antibody comprising a heavy chain variable region comprising the sequence of SEQ ID NO: 7, and a light chain variable region comprising the sequence of any of SEQ ID NO: 8.

Antibody Variants

Antibody 5-10 and antigen-binding fragments thereof provided herein also encompass various variants of the antibody sequences provided herein.

In certain embodiments, the antibody variants comprise one or more mutations in one or more of the CDR sequences provided in Table 1 above, one or more of the non-CDR sequences of the heavy chain variable region or light chain variable region provided in Table 2 above, and/or the constant region (e.g. Fc region). Such variants retain binding specificity to spike protein of SARS-CoV-2 of their parent antibodies, but have one or more desirable properties conferred by the mutation(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example, “alanine scanning mutagenesis” (see, for example, Cunningham and Wells (1989) Science, 244:1081-1085). Briefly, target residues (e.g. charged residues such as Arg, Asp, His, Lys, and Glu) can be identified and replaced by a neutral or negatively charged amino acid (e.g. alanine or polyalanine), and the modified antibodies are produced and screened for the interested property. If substitution at a particular amino acid location demonstrates an interested functional change, then the position can be identified as a potential residue for mutation. The potential residues may be further assessed by substituting with a different type of residue (e.g. cysteine residue, positively charged residue, etc.).

Affinity Variants

Affinity variants of antibodies may contain mutations in one or more CDR sequences provided in Table 1 above, the heavy or light chain variable region sequences provided in Table 2, or one or more FR sequences which can be readily identified by a person skilled in the art based on the CDR sequences provided in Table 1 and the heavy or light chain variable region sequences provided in Table 2, as it is well-known in the art that a CDR region is flanked by two FR regions in the variable region. The affinity variants retain specific binding affinity to spike protein of SARS-CoV-2 of the parent antibody, or even have improved spike protein of SARS-CoV-2 specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Table 1 above, and variable region sequences provided in Table 2 above, one or more amino acid residues may be substituted yet the resulting antibody or antigen-binding fragment still retain the binding affinity or binding capacity to spike protein of SARS-CoV-2, or even have an improved binding affinity or capacity. Various methods known in the art can be used to achieve this purpose. For example, a library of antibody variants (such as Fab or scFv variants) can be generated and expressed with phage display technology, and then screened for the binding affinity to spike protein of SARS-CoV-2. For another example, computer software can be used to virtually simulate the binding of the antibodies to spike protein of SARS-CoV-2, and identify the amino acid residues on the antibodies which form the binding interface. Such residues may be either avoided in the substitution so as to prevent reduction in binding affinity, or targeted for substitution to provide for a stronger binding.

In certain embodiments, the antibodies and antigen-binding fragments thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the antibodies and antigen-binding fragments thereof provided herein comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Table 1 above yet retaining the specific binding to spike protein of SARS-CoV-2 at a level similar to or even higher than its parent antibody.

In certain embodiments, the antibodies and antigen-binding fragments thereof provided herein comprise one or more variable region sequences having at least 80% (e.g. at least 85%, 88%, 90%, 91%, 92%, 93%, 94%, 95%, 96%, 97%, 98%, 99%) sequence identity to that (or those) listed in Table 2 above yet retaining the specific binding affinity to spike protein of SARS-CoV-2 at a level similar to or even higher than its parent antibody. In some embodiments, the mutations occur in regions outside the CDRs (e.g. in the FRs).

Glycosylation Variants

The antibodies and antigen-binding fragments thereof provided herein also encompass glycosylation variants, which can be obtained to either increase or decrease the extent of glycosylation of the antibodies or antigen binding fragments thereof.

The antibodies or antigen binding fragments thereof may comprise one or more modifications that introduce or remove a glycosylation site. A glycosylation site is an amino acid residue with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

Cysteine-Engineered Variants

The antibodies and antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

Fc Variants

The antibodies and antigen-binding fragments thereof provided herein also encompass Fc variants, which comprise one or more amino acid residue mutations at the Fc region and/or hinge region, for example, to provide for altered effector functions such as ADCC and CDC. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O., et al., Mol Cancer Ther. 2008, 7(8): 2517-27; Shields R. L. et al., J. Biol. Chem, 2002, 277: 26733-26740; Shinkawa T. et al., J. Biol. Chem, 2003, 278: 3466-3473.

CDC activity of the antibodies or antigen-binding fragments provided herein can also be altered, for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fe region variants.

One or more amino acids selected from amino acid residues 329, 331 and 322 of the Fc region can be replaced with a different amino acid residue to alter C1q binding and/or reduced or abolished complement dependent cytotoxicity (CDC) (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

Also encompassed herein are antibodies and antigen-binding fragments thereof provided herein having Fc variants with one or more amino acid residue mutations at the Fc region and/or hinge region, to provide for reduced or eliminated antibody dependent enhancement (ADE) of SARS-CoV-2 infection. Such Fc variants may have reduced binding to Fc receptors (FcR). Examples of such mutations include, without limitation, mutations of leucine residues at positions 4, 5, or both of CH2 domain (e.g. to alanine, as LALA variant), see, for example, WO2010043977A2, which is incorporated herein to its entirety.

Antigen-Binding Fragments

Provided herein are also neutralizing antigen-binding fragments against spike protein of SARS-CoV-2. Various types of antigen-binding fragments are known in the art and can be developed based on the neutralizing antibodies against spike protein of SARS-CoV-2 provided herein, including for example, the exemplary antibodies whose CDR are shown in Table 1 above, and variable sequences are shown in Table 2, and their different variants (such as affinity variants, glycosylation variants, Fc variants, cysteine-engineered variants and so on).

In certain embodiments, a neutralizing antigen-binding fragments against spike protein of SARS-CoV-2 provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

Various techniques can be used for the production of such antigen-binding fragments. Illustrative methods include, enzymatic digestion of intact antibodies (see, e.g. Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, antibodies and antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

Bispecific or Multispecific Antibodies

In certain embodiments, the antibodies and antigen-binding fragments thereof provided herein is bispecific or multispecific. In certain embodiments, the antibody or antigen-binding fragment thereof is further linked to a second functional moiety having a different binding specificity from said antibodies, or antigen binding fragment thereof.

In certain embodiments, the bispecific or multispecific antibodies or antigen-binding fragments thereof provided herein comprises a combination of two or more of the antigen-binding fragments. In certain embodiments, the two or more of the antigen-binding fragments in the bispecific or multispecific antibodies or antigen-binding fragments thereof provided herein bind to distinct epitopes on spike protein of the SARS-CoV-2 or distinct antigens of SARS-CoV-2. In some embodiments, the bispecific antibody or antigen-binding fragment thereof provided herein is capable of specifically binding to distinct epitopes on S1 subunit of spike protein of SARS-CoV-2 or distinct subunits of spike protein of SARS-CoV-2. In certain embodiments, the two or more of the antibodies or antigen-binding fragments thereof in the bispecific antibodies or antigen-binding fragments thereof provided herein specifically bind to SARS-CoV-2 in a non-competing manner.

In certain embodiments, the bispecific or multispecific antibodies or antigen-binding fragments thereof further comprises a second antigen-binding fragment.

In certain embodiments, the bispecific or multispecific antibodies or antigen-binding fragments thereof further comprises a second antigen-binding fragment and a third antigen-binding fragment.

In certain embodiments, the bispecific or multispecific antibodies or antigen-binding fragments thereof provided herein comprises a first antigen-binding fragment provided herein, and a second antigen-binding fragment capable of neutralizing SARS-CoV-2. In certain embodiments, the second antigen-binding fragment is capable of binding to SARS-CoV-2 at an epitope distinct from that/those bound by the antibodies or antigen-binding fragments provided herein. In certain embodiments, the second antigen-binding fragment is capable of binding to spike protein of the SARS-CoV-2 at an epitope different from that/those bound by Antibody Antibody 5-10.

In certain embodiments, the second antigen-binding fragment is capable of binding to non-RBD region of spike protein of the SARS-CoV-2.

In certain embodiments, the bispecific or multispecific antibodies or antigen-binding fragments thereof provided herein are capable of specifically binding to a second antigen other than spike protein of SARS-CoV-2, or a second epitope on spike protein of SARS-CoV-2.

Without wishing to be bound by theory, it is believed that bispecific antibodies (bsAbs) without Fc may have a relatively shorter half-life but higher tissue penetration rate than bsAbs with Fc, and that bsAbs with Fc have better stability with retention of Fc associated physiological characteristics and biological activity. In certain embodiments, the antibodies or antigen-binding fragments thereof provided herein is a bispecific antibody (bsAb) with Fc region. In certain embodiments, the antibodies or antigen-binding fragments thereof provided herein is a bispecific antibody without Fc region.

BsAbs may be in various formats, ranging from small proteins with merely two linked antigen-binding fragments to IgG-like molecules with additional domain attached, detailed of which are described in Aran F. Labrijn et al., Nature Reviews Drug Discovery 18(8), 585-608 (2019).

Combination of Antibodies

In another aspect, the present disclosure provides a composition comprising a combination of one or more antibodies and antigen-binding fragments. In certain embodiments, the combination comprises the antibodies and antigen-binding fragment thereof, binding to distinct epitopes on spike protein of the SARS-CoV-2.

In certain embodiments, the pharmaceutical composition comprises a combination of two or more of the antibodies or antigen-binding fragments thereof. In certain embodiments, the two or more of the antibodies or antigen-binding fragments thereof in the combination bind to distinct epitopes on spike protein of the SARS-CoV-2. In certain embodiments, the two or more of the antibodies or antigen-binding fragments thereof in the combination specifically bind to SARS-CoV-2 in a non-competing manner.

Studies have shown that applying antibodies against RBD of spike protein of SARS-CoV-2 individually may induce resistance mutations in the SARS-CoV-2 (see, for example, Zhou et al., Structural definition of a neutralization epitope on the N-terminal domain of MERS-CoV spike glycoprotein. Nat. Commun. 10, 3068 (2019)). Use of a cocktail of antibodies against different antigens and/or epitopes, is generally considered as an effective way to solve the problem caused by such resistance mutations in viruses, e.g., reduction in or loss of effectiveness of the neutralizing antibodies. Accordingly, combinatory use of antibodies against RBD of spike protein of SARS-CoV-2 with antibodies against non-RBD of spike protein of SARS-CoV-2 or use of bispecific or multispecific antibodies described above is more advantageous in treating and/or preventing SARS-CoV-2 infection.

Conjugates

In some embodiments, the antibody and antigen-binding fragments thereof provided herein further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibody or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding, affinity binding, intercalation, coordinate binding, complexation, association, blending, or addition, among other methods.

In certain embodiments, the antibody or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies or antigen-binding fragments thereof may be linked to a conjugate moiety indirectly, or through another conjugate moiety. For example, the antibody or antigen-binding fragments thereof provided herein may be conjugated to biotin, then indirectly conjugated to a second conjugate that is conjugated to avidin. In some embodiments, the conjugate moiety comprises a clearance-modifying agent (e.g. a polymer such as PEG which extends half-life), a detectable label (e.g. a luminescent label, a fluorescent label, an enzyme-substrate label), or other therapeutic molecules.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moieties, digoxigenin, biotin/avidin, DNA molecules or gold for detection.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibody or antigen-binding fragments thereof provided herein is used as a base for a conjugate.

Polynucleotides and Recombinant Methods

The present disclosure provides isolated polynucleotides that encode the antibody or antigen-binding fragments thereof provided herein.

In some embodiments, the isolated polynucleotides encodes a heavy chain variable region and comprise a sequence consisting of: SEQ ID NO: 9 and a sequence having at least 80% (e.g. at least 85%, 88%, 90%, 91%, 92%, 93%, 94%, 95%, 96%, 97%, 98%, 99%) sequence identity thereof. In some embodiments, the isolated polynucleotides encodes a light chain variable region and comprise a sequence consisting of: SEQ ID NO: 10 and a homologous sequence thereof having at least 80% (e.g. at least 85%, 88%, 90%, 91%, 92%, 93%, 94%, 95%, 96%, 97%, 98%, 99%) sequence identity. In certain embodiments, the percentage identity is due to genetic code degeneracy, while the encoded protein sequence remains unchanged.

The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g. by using oligonucleotide probes that are capable of binding specifically to genes encoding the heavy and light chains of the antibody). In certain embodiments, DNA encoding the monoclonal antibody is isolated and sequenced using high throughput next generation sequencing techniques. The encoding DNA may also be obtained by synthetic methods.

The isolated polynucleotide that encodes the antibody or antigen-binding fragments thereof provided herein can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

In certain embodiments, the expression vector comprises a viral vector or a non-viral vector. Examples of viral vectors include, without limitation, adeno-associated virus (AAV) vector, lentivirus vector, retrovirus vector, and adenovirus vector. Examples of non-viral vectors include, without limitation, naked DNA, plasmid, exosome, mRNA, and so on. In certain embodiments, the expression vector is suitable for gene therapy in human. Suitable vectors for gene therapy include, for example, adeno-associated virus (AAV), or adenovirus vector. In certain embodiments, the expression vector comprises a DNA vector or an RNA vector. In certain embodiments, the pharmaceutically acceptable carriers are polymeric excipients, such as without limitation, microspheres, microcapsules, polymeric micelles and dendrimers. The polynucleotides, or polynucleotide vectors of the present disclosure may be encapsulated, adhered to, or coated on the polymer-based components by methods known in the art (see for example, W. Heiser, Nonviral gene transfer techniques, published by Humana Press, 2004; U.S. Pat. No. 6,025,337; Advanced Drug Delivery Reviews, 57(15): 2177-2202 (2005)).

The present disclosure provides vectors comprising the isolated polynucleotides provided herein. In certain embodiments, the polynucleotide provided herein encodes the antibodies or antigen-binding fragments thereof, at least one promoter (e.g. SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheruformis, Pseudomonas such as P. aeruginosa, and Streptomyces.

In addition to prokaryotes, eukaryotic microbes such as filamentous fungi or yeast are suitable cloning or expression hosts for the vectors provided herein. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibody or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g. the L−1 variant of Autographa calfornica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for production of the neutralizing antibodies against spike protein of SARS-CoV-2 provided herein and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or an antigen-binding fragment thereof provided herein, comprising culturing the host cell provided herein under the condition at which the vector of the present disclosure is expressed. The host cells used to produce the antibody or antigen-binding fragments thereof provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The neutralizing antibodies against spike protein of SARS-CoV-2 or antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g. from about 0-0.25M salt).

Polynucleotide Composition

The present disclosure further provides compositions comprising a first mRNA polynucleotide encoding heavy chain or an antigen-binding fragment thereof of the antibody provided herein (e.g. Antibody 5-10), and a second mRNA polynucleotide encoding light chain or a fragment thereof of the antibody provided herein (e.g. Antibody 5-10).

In certain embodiments, the mRNA polynucleotide further comprises a nucleotide sequence encoding a signal peptide. With respect to the first mRNA polynucleotide, the signal peptide can be operably linked to the heavy chain or an antigen-binding fragment thereof. Similarly, with respect to the second mRNA polynucleotide, the signal peptide can be operably linked to the light chain or an antigen-binding fragment thereof. Signal peptide is typically present at N-terminal of a newly synthesized protein, and can be removed proteolytic cleavage.

mRNA polynucleotides can be synthesized using any suitable methods known in the art, for example, by in vitro transcription (IVT), which involves synthesizing mRNA using a suitable DNA template containing a promoter, an RNA polymerase, a mixture of ribonucleotide triphosphates, suitable buffer, among others.

The mRNA can be unmodified or modified, for example, to improve stability. A variety of modification can be useful, for example, modifications on RNA backbone, nucleobase, sugar, or phosphate linkage.

In certain embodiments, the mRNA polynucleotide further comprises a 5′ cap structure and/or a 3′ tail structure such as poly(A) or poly(C).

In certain embodiments, the mRNA polynucleotide further comprises a 5′ and/or 3′ untranslated region, which may include, for example, one or more elements that are useful to improve stability or translation of the protein-encoding sequence.

In certain embodiments, the composition further comprises a pharmaceutically acceptable carrier.

In certain embodiments, the pharmaceutically acceptable carrier can be a carrier suitable for delivering the mRNA polynucleotide. Such carriers may include, for example, polymer-based carriers, lipid-based carriers, or any combination thereof. Polymer-based carriers may form nanoparticles or microparticles, or may be protein or polypeptides that are useful for delivery of mRNA. Lipid-based carriers may include, for example, cationic lipids, non-cationic lipids, PEG-modified lipids, and so on.

In another aspect, the present disclosure provides a method of producing the antibody provided herein, and the method comprises administering the polynucleotide composition provided herein to a cell, wherein the first mRNA polynucleotide and the second mRNA polynucleotide are expressed in the cell, thereby producing the antibody.

In another aspect, the present disclosure provides a method of delivering an antibody provided herein in a subject, and the method comprises administering the composition provided herein to a subject in need thereof, wherein the first mRNA polynucleotide and the second mRNA polynucleotide are expressed in the cell, thereby producing the antibody.

Pharmaceutical Composition

The present disclosure further provides pharmaceutical compositions comprising a neutralizing antibody against spike protein of SARS-CoV-2 or antigen-binding fragments thereof and one or more pharmaceutically acceptable carriers.

The present disclosure further provides pharmaceutical compositions comprising a combination of two or more antibodies or antigen-binding fragments thereof. In certain embodiments, the two or more of the antibodies or antigen-binding fragments thereof in the combination bind to distinct epitopes on spike protein of the SARS-CoV-2. In certain embodiments, the two or more of the antibodies or antigen-binding fragments thereof in the combination bind to distinct epitopes on S1 subunit of spike protein of SARS-CoV-2 or distinct subunits of spike protein of SARS-CoV-2. In certain embodiments, the two or more of the antibodies or antigen-binding fragments thereof in the combination specifically bind to SARS-CoV-2 in a non-competing manner.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants, such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the neutralizing antibody against spike protein of SARS-CoV-2 or antigen-binding fragment thereof or composition thereof. Overfilling vials with a small amount above that needed for a dose or set of doses (e.g. about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

Kits

In certain embodiments, the present disclosure provides a kit comprising the antibody or an antigen-binding fragment thereof provided herein, or a combination of two or more of the antibodies or antigen-binding fragments thereof, and/or the pharmaceutical composition provided herein. In certain embodiments, the present disclosure provides a kit comprising the antibody or an antigen-binding fragment thereof provided herein, and a second therapeutic agent. The second therapeutic agent can be a second SARS-CoV-2 neutralizing antibody, an antiviral agent such as RNA dependent RNA polymerase inhibitor, a nucleoside analog, antiviral cytokines (such as interferons), immunostimulatory agents, and other antiviral agents.

In certain embodiments, the second SARS-CoV-2 neutralizing antibody can be any antibody that has neutralizing activity on SARS-CoV-2, and optionally binds to an epitope that is different from those/that bound by the antibodies provided herein. Examples of neutralizing antibodies include, those reported in the publications for example, Cao, Y. et al (2020). Cell, doi: 10.1016/j.cell.2020.05.025; Ju, B., et al., (2020). Nature. https://doi.org/10.1038/s41586-020-2380-z; Pinto, D. et al, (2020). Nature. 2020 May 18. doi: 10.1038/s41586-020-2349-y.; Shi, R. et al, (2020). Nature, (2020). https://doi.org/10.1038/s41586-020-2381-y; Wang, C. et al, (2020). Nat Commun 11(1): 2251; Wu, Y. et al, (2020). Science 368(6496): 1274-1278, which are incorporated herein by reference.

In certain embodiments, the second therapeutic agent is selected from the group consisting of Ivermectin, Colcrys (colchicine), Avigan (favipiravir) and other antiviral medications, Tamiflu (oseltamivir), Kaletra (lopinavir/ritonavir), Actemra (tocilizumab), Convalescent plasma, Azithromycin, Hydroxychloroquine and chloroquine, Dexamethasone, Remdesivir, Fluvoxamine, Bevacizumab, sarilumab, Tocilizumab, Corticosteroids, Nitazoxanide, Umifenovir, Famotidine, camostat, and Nafamostat.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

Methods of Use

In one aspect, the present disclosure also provides methods of treating SARS-CoV-2 infection in a subject, comprising administering to the subject an effective amount of the antibody or antigen-binding fragment thereof provided herein, and/or the pharmaceutical composition provided herein.

In another aspect, the present disclosure also provides methods for preventing, inhibiting progression of, and/or delaying the onset of SARS-CoV-2 infection or a SARS-CoV-2-associated condition in a subject, comprising administering to the subject an effective amount of the antibody or antigen-binding fragment thereof provided herein, and/or the pharmaceutical composition provided herein, and/or the polynucleotide composition provided herein.

In another aspect, the present disclosure also provides methods for preventing or reducing transmission of SARS-CoV-2 by a SARS-CoV-2 infected subject, comprising administering to the subject an effective amount of the antibody or antigen-binding fragment thereof provided herein, and/or the pharmaceutical composition provided herein, and/or the polynucleotide composition provided herein.

In some embodiments, the present disclosure also provides methods for reducing viral load in a SARS-CoV-2 infected subject, comprising administering to the subject an effective amount of the antibody or antigen-binding fragment thereof provided herein, and/or the pharmaceutical composition provided herein, and/or the polynucleotide composition provided herein.

The present disclosure also provides methods of neutralizing SARS-CoV-2 in a subject therewith.

In certain embodiments, the subject is human.

In certain embodiments, the subject is a human with or at risk for SARS-CoV-2 infection. SARS-CoV-2 infection can include, for example, infection of SARS-CoV-2 at respiratory tract, including nasal cavity infection, lower respiratory tract infection, or lung infection.

In certain embodiments, the subject is human exposed to or suspected of having exposure to SARS-CoV-2. The term “SARS-CoV-2 exposure” means being exposed to an environment where a SARS-CoV-2 carrier is present or has appeared. A “SARS-CoV-2 carrier” refers to any living or non-living subject with transmissible SARS-CoV-2 on or in it. “Transmissible SARS-CoV-2” refers to SARS-CoV-2 capable of spreading from one living or non-living subject to another living or non-living subject.

The term “effective amount” as used herein refers to a dosage of a medicament which can significantly eliminating, ameliorating or improving the symptoms associated with a disease or abnormal condition or which can produce the desired effect of preventing onset of symptoms associated with a disease or abnormal condition or even preventing the development of a disease or abnormal condition. The disease or abnormal condition can be associated with viral infection, such as SARS-CoV-2 infection. The effective amount of the antibodies or antigen binding fragment thereof of the present disclosure means the dosage thereof that can result in eliminating, ameliorating or improving symptoms associated with onset of SARS-CoV-2 infection symptoms, including but is not limited to, fever or chills, cough, shortness of breath or difficulty breathing, fatigue, muscle or body aches, headache, new loss of taste or smell, sore throat, congestion or runny nose, nausea or vomiting, and diarrhea; the effective amount of the antibodies or antigen binding fragment thereof of the present disclosure also means the dosage thereof that can effectively prevent SARS-CoV-2 infection or effectively prevent onset of SARS-CoV-2 infection symptoms.

The effective amount of an antibody or antigen-binding fragment provided herein will depend on various factors known in the art, such as body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g. physician or veterinarian) as indicated by these and other circumstances or requirements.

In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g. a therapeutic response). For example, a single dose may be administered, or several divided doses may be administered over time.

The antibody or antigen-binding fragments thereof provided herein may be administered by any route known in the art, such as for example parenteral (e.g. subcutaneous, intraperitoneal, intravenous, including intravenous infusion, intramuscular, or intradermal injection) or non-parenteral (e.g. oral, intranasal, intraocular, sublingual, rectal, or topical) routes.

In some embodiments, the antibody or antigen-binding fragments thereof provided herein may be administered alone or in combination with a therapeutically effective amount of a second therapeutic agent. For example, the antibodies or antigen-binding fragments thereof disclosed herein may be administered in combination with a second therapeutic agent, for example, a second SARS-CoV-2 neutralizing antibody, an antiviral agent such as RNA dependent RNA polymerase inhibitor, a nucleoside analog, antiviral cytokines (such as interferons), immunostimulatory agents, and other antiviral agents.

In certain of these embodiments, an antibody or antigen-binding fragment thereof provided herein that is administered in combination with one or more additional therapeutic agents may be administered simultaneously with the one or more additional therapeutic agents, and in certain of these embodiments the antibody or antigen-binding fragment thereof and the additional therapeutic agent(s) may be administered as part of the same pharmaceutical composition. However, an antibody or antigen-binding fragment thereof administered “in combination” with another therapeutic agent does not have to be administered simultaneously with or in the same composition as the agent. An antibody or antigen-binding fragment thereof administered prior to or after another agent is considered to be administered “in combination” with that agent as the phrase is used herein, even if the antibody or antigen-binding fragment and the second agent are administered via different routes. Where possible, additional therapeutic agents administered in combination with the antibodies or antigen-binding fragments thereof disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

In another aspect, the present disclosure provides methods of detecting the presence or amount of spike protein of SARS-CoV-2 in a sample, comprising contacting the sample with the antibody or antigen-binding fragment thereof provided herein and/or the pharmaceutical composition provided herein, and determining the presence or the amount of spike protein of SARS-CoV-2 in the sample.

In another aspect, the present disclosure provides a method of diagnosing SARS-CoV-2 infection in a subject, comprising: a) contacting a sample obtained from the subject with the antibody or an antigen-binding fragment thereof provided herein and/or the pharmaceutical composition provided herein; b) determining the presence or amount of spike protein of SARS-CoV-2 in the sample; and c) correlating the presence or the amount of spike protein of SARS-CoV-2 to existence or status of SARS-CoV-2 virus in the subject.

In another aspect, the present disclosure provides kits comprising the antibody or antigen-binding fragment thereof provided herein and/or the pharmaceutical composition provided herein, optionally conjugated with a detectable moiety, which is useful in detecting SARS-CoV-2 virus. The kits may further comprise instructions for use.

In another aspect, the present disclosure also provides use of the antibody or antigen-binding fragment thereof provided herein and/or the pharmaceutical composition provided herein, and/or the polynucleotide composition provided herein in the manufacture of a medicament for treating or preventing SARS-CoV-2 infection in a subject; or for preventing, inhibiting progression of, and/or delaying the onset of SARS-CoV-2 infection or a SARS-CoV-2-associated condition in a subject; or for preventing or reducing transmission of SARS-CoV-2 by a SARS-CoV-2 infected subject; or for reducing viral load in a SARS-CoV-2 infected subject.

In another aspect, the present disclosure also provides use of the antibody or antigen-binding fragment thereof provided herein and/or the pharmaceutical composition provided herein in the manufacture of a diagnostic reagent for diagnosing SARS-CoV-2 infection.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Generation of Recombinant Monoclonal Antibodies Against SARS-CoV-2

Blood samples from 20 patients were obtained and antibodies against SARS-CoV-2 were detected by ELISA. To obtain SARS-CoV-2 specific monoclonal Abs (mAbs), convalescent patients' memory B cells were stained with a fluorescently labeled SARS-CoV-2 ACE2 receptor binding domain (RBD) protein and sorted by flow cytometry. Transcriptionally-active PCR was used to generate individual clones from thousands of single sorted B cells rapidly. Thousands of antibody clones were directly transfected into CHO cell line for antibody screening. 1554 antibodies binding to SARS-CoV-2 RBD by ELISA were observed. A blocking assay were established to screen antibodies that can block binding between ACE2 and RBD. It was observed that a total of 114 out of 1554 RBD antibodies significantly blocked binding between ACE2 and RBD. Antibody 5-10 with Kd of 4.314 nM was selected, which showed potent neutralizing effects on SARS-CoV-2 pseudovirus, with IC50 of 0.0425 ug/mL.

Methods

Assessment of the RBD-Specific Antibody by Enzyme-Linked Immunosorbent Assay (ELISA)

SARS-CoV-2 RBD (Vazyme Biotech, SEQ ID NO: 11) was diluted to final concentrations of 0.5-1 g/mL, followed by being coated onto 96-well plates and incubated at 4° C. overnight. The 96-well plates were washed with PBS-T twice and blocked with blocking buffer (PBS containing 5% BSA) at 37° C. for 2 h. Diluted plasma samples from convalescent patients or mAbs were added to the plates and incubated at 37° C. for 1 h. Wells were then incubated with secondary anti-human IgG labeled with HRP and TMB substrate. Optical density (OD) was measured by a spectrophotometer at 450 nm.

Sorting of RBD-Specific Single B Cells by FACS

PBMCs were stained with cocktail consisted of CD27-APC, IgG-PE, IgM-PerCP-Cy5.5 and the recombinant RBD-FITC. RBD-specific single B cells were gated as CD27+IgM-IgG+RBD+ and sorted into 96-well PCR plates containing lysis buffer (Vazyme Biotech). Plates were then snap-frozen on dry ice and stored at −80° C. until room temperature (RT) reaction.

Rapid Generation of Recombinant Functional Monoclonal Antibodies from Single B Cells

Antibody variable-region genes were then recovered via two rounds of PCR using DNA polymerase (Vazyme Biotech). A primary PCR utilized gene-specific primers at both the 5′ and 3′ ends. Not only did the secondary oligonucleotides introduce restriction sites to facilitate downstream cloning, but they also provided ˜25 base-pair overlap regions; at the 5′ end with a human cytomegalovirus (HCMV) promoter fragment (plus a leader sequence for rat-derived fragments that were generated with the framework 1 primer set) and at the 3′ end with a heavy or light chain constant region fragment. Then, in a tertiary PCR, variable region DNA, HCMV promoter fragment and constant region fragment containing a poly-adenylation sequence were combined and amplified to produce two separate linear transcriptionally active PCR (TAP) products, one encoding the heavy chain and the other the light chain. Variable regions were recombined with constant regions in the expression cassettes to produce monoclonal antibodies.

Antibody 5-10 was obtained. The amino acid sequences and nucleic acid sequences of the monoclonal Antibody 5-10 are shown in Tables 2 and 3 of the present disclosure.

Further engineering on the antibodies are ongoing, and a number of mutations are made to replace the amino acid residues back to germ line sequences. It is expected to make the candidate antibodies more “humaness” and less immunogenic.

### Example 2. Characterization of the Recombinant Monoclonal Antibody Against SARS-CoV-2

ELISA Analysis of Antibody 5-10 Binding to RBD of the Spike Protein of SARS-CoV-2 (SARS-CoV-2 RBD)

SARS-CoV-2 RBD (Vazyme Biotech) was diluted to final concentrations of 4 g/mL, then coated onto 96-well plates and incubated at 37° C. for 2 h. Samples were washed with PBS-T three times and blocked with blocking buffer (PBS containing 5% BSA) at 37° C. for 2 h. Diluted Antibody 5-10 was added the plates and incubated at 37° C. for 1 h. Wells were then incubated with secondary anti-human IgG labeled with HRP and TMB substrate. Optical density (OD) was measured by a spectrophotometer at 450 nm.

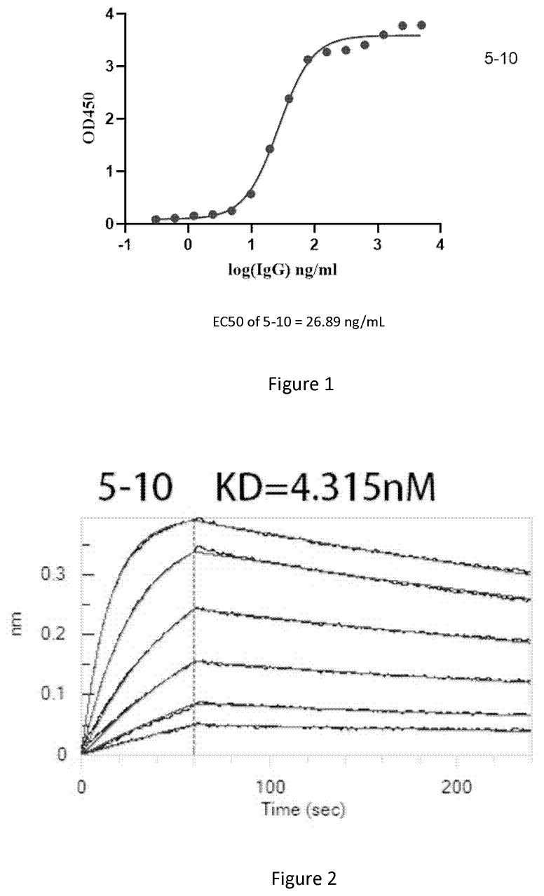

As can be seen in FIG. 1, Antibody 5-10 exhibit specific binding to SARS-CoV-2 RBD.

Antibodies Affinity Characterization

Binding kinetics of anti-SARS-CoV-2 RBD Antibody 5-10 were determined using biolayer interferometry on a ForteBio Octet RED96e. Biosensors were coupled with SARS-CoV-2 RBD (100 nM to 3.13 nM) for 60 S. The Biosensors dissociate in Sample Dilution Buffer for 180 s. Binding kinetics was evaluated using a 1:1 Langmuir binding model by ForteBio Data Analysis 185 7.0 software.

As can be seen in FIGS. 2, Antibody 5-10 showed excellent binding affinity to SARS-CoV-2 RBD, with a KD value of 4.315 nM.

RBD-ACE Blocking Assay

The HTRF ACE2/RBD Binding Assay is designed to measure the interaction between ACE2 and RBD. Utilizing HTRF (Homogeneous Time-resolved Fluorescence) technology, the assay enables simple and rapid characterization of compound and antibody blockers in a high throughput format. The interaction between ACE2 and RBD is detected by using differently tagged RBD and ACE2. When antibodies are brought into close proximity due to ACE2 and RBD binding, excitation of the donor antibody triggers fluorescence resonance energy transfer (FRET) towards the acceptor antibody, which in turn emits specifically at 665 nm. This specific signal is directly proportional to the extent of ACE2/RBD interaction. Thus, antibodies blocking ACE2/RBD interaction will cause a reduction in HTRF signal.

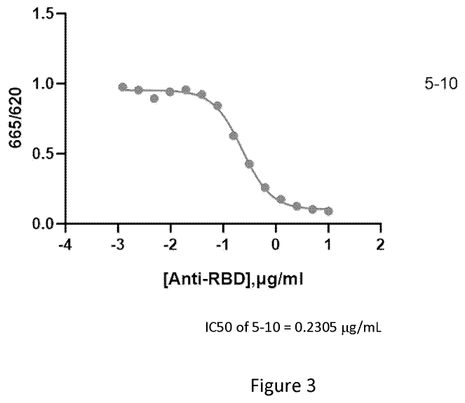

As can be seen in FIG. 3, Antibody 5-10 at a concentration as low as no more than 0.3 μg/mL can almost completely block the binding of SARS-CoV-2 RBD to ACE2, suggesting excellent SARS-CoV-2 RBD blocking capability of the neutralizing antibodies or antigen-binding fragment thereof of the present disclosure.

Pseudovirus Blocking Assay

SARS-CoV-2 S pseudotyped virus neutralization assay were performed as described previously (Matsuyama, S. et al., (2018). J. Virol. 92, e00683-18). Briefly, 5-fold dilution Antibody 5-10 was incubated with the same volume of SARS-COV-2 pseudovirus with a TCID50 of 1.3×104 for 1 h at 37° C. The mixtures were then used to infect Huh7 cells seeded in 96-well plates for 24 h at 37° C. After the incubation, supernatants were removed, and Luciferase was added to each well and incubated for 2 mins. After the incubation, luciferase activities were measured using a microplate spectrophotometer (PerkinElmer EnSight). The inhibition rate is calculated by comparing the OD value to the negative and positive control wells. IC50 were determined by a four-parameter logistic model using GraphPad Prism 7.0.

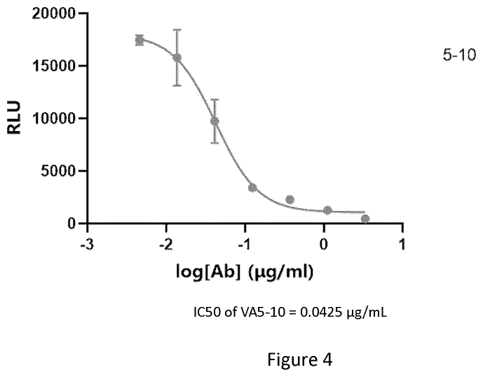

The results are shown in FIG. 4. Antibody 5-10 showed dose-dependent neutralization of pseudotyped virus, and the IC50 value is about 40 ng/mL.

Animal Study

A human ACE2 humanized mouse is used for the study of the antiviral activity of the monoclonal antibodies. Generation of such hACE2 humanized mice was described in Sun et al., Cell Host & Microbe (2020), https://doi.org/10.1016/j.chom.2020.05.020. Briefly, hACE2 gene is inserted into the first coding exon of mACE2 by homologous recombination. The insertion of inserted hACE2 is confirmed by PCR. The generated hACE2 humanized mice are divided into blank control groups (with no virus challenge), negative control groups (with virus challenge but without any treatment), and treatment groups (with virus challenge and with different doses of testing antibodies). Antiviral effects are observed in the hACE2 humanized mice. It is expected that Antibody 5-10 show good antiviral effects in the hACE2 humanized mice model and protects the mice from infecting SARS-CovV-2 or alleviates the symptoms or disease development of SARS-CovV-2 infection.

### Example 3. Further Characterization of Antibody 5-10

The luciferase reporter gene system was used to detect the blocking effect of Antibody 5-10 against SARS-CoV-2 wild-type infection on 293-ACE2 cells. First, wild-type (2 μl virus/well) was pre-incubated with Antibody 5-10 or control antibody (from 0.1 μg/ml to 100 μg/ml, 10-fold dilution) at 37° C. for 1 h. Then the 293-ACE2 cells were resuspended in the buffer, and 20,000 cells were added to the mixture of pseudovirus and antibody in each well and incubated in a 37° C. incubator for 24 h. After the incubation, 50 μl Bright light Luciferase substrate was added to each well, fluorescence signal was detected the with a microplate reader, and GraphPad Prism software was used to perform a four-parameter fitting curve to obtain the IC50 value.

On 293-ACE2 cells, the luciferase reporter gene system was used to detect the neutralizing activity of Antibody 5-10 injection against pseudovirus. Results are illustrated in FIG. 5. Antibody 5-10 can effectively neutralize the wild-type SARS-CoV-2 pseudovirus with an IC50 value of 15.33 ng/ml.

JS016 is the first fully humanized anti-SARS-CoV-2 monoclonal antibody injection (IgG1K) independently developed by Junshi Bio, and has been under clinical trials. The amino acid sequences of VH and VL of the JS016 antibody are published in WO2021169932A1, and are included in the present application as SEQ ID NOs: 15 and 16. Results of pseudovirus neutralization study show that JS016 exhibit relatively significant neutralizing activity against the wild-type SARS-CoV-2, with an IC50 value of 63.07 ng/ml, and the neutralizing activity is significantly lower than Antibody 5-10.

In conclusion, Antibody 5-10 injection can effectively neutralize the SARS-CoV-2 pseudovirus, and exhibits strong viral inhibitory activity against wild-type SARS-CoV-2 infection on 293-ACE2 cells, therefore Antibody 5-10 injection was supposed to prevent wild-type SARS-CoV-2 from infecting humans.

### Example 4. Live Virus Neutralizing Activity Study In Vivo

The hACE2 transgenic mice (female, 30 weeks) were divided into 3 groups, 8 animals in each group, as shown in Table 4. In the prophylaxis group, the mice were intraperitoneally injected with 20 mg/kg of Antibody 5-10 one day before the nasal challenge of SARS-CoV-2 virus. Antibody treatment group received intraperitoneal injection of 20 mg/kg Antibody 5-10 one day after challenge. Placebo group received intraperitoneal injection of 100 μL sterile PBS one day after SARS-CoV-2 virus challenge. On the 5th day after the challenge, all mice were euthanized and lung tissues were collected. 5 of the 8 mice in each group were examined for lung viral load, and the 3 residual mice were fixed with 4% paraformaldehyde for HE staining in observation with pulmonary pathology.

TABLE 4
Experiment group design

| Group | Number | Dosage regimen |
|---|---|---|
| Prophylaxis group | 8 | Antibody 5-10: 20 mg/kg |
| Treatment group | 8 | Antibody 5-10: 20 mg/kg |
| Placebo group | 8 | PBS |

The lung tissue used for lung viral load detection was added with 1.5 mL DMEM medium and ground, centrifuged at 5000 g/min for 10 min, and the supernatant was collected for RNA extraction. 140 μL supernatant was taken from each sample, and RNA was extracted using QIAamp viral RNA mini kit. Primers targeting SARS-CoV-2 ORF1ab gene were used.

According to the instructions, the copy number of RNA in the sample was determined by using the One Step PrimeScript™ RT-PCR Kit (Perfect Real Time) (Takara Co., Ltd), and a standard curve was drawn by using the standard plasmid solution of the ORF1ab gene in gradient dilutions. Reverse transcription was performed at 42° C. for 5 min on the ABI QuantStudio 5 fluorescent quantitative PCR instrument. Pre-denaturation at 95° C. for 10 s, 40 cycles comprising denaturation at 95° C. for 3 s, and annealing extension at 60° C. for 30 s. The CT values of each sample is converted to virus copy number automatically by instruments according to standard curve. Finally, viral load of mouse lungs was calculated based on viral copy number and lung weight of each mouse (copies/g).

The RBD region of the S protein of SARS-CoV-2 can bind to the ACE2 receptor on the surface of human cells to mediate the virus intrusion into the target cells in human body, thereby causing the virus to infect humans. The fully human monoclonal antibody Antibody 5-10 can specifically bind to RBD, thereby blocking RBD-ACE2 binding, inhibiting virus intrusion and infecting target cells, and realizing treating pneumonia with SARS-CoV-2 infection. The monoclonal antibody Antibody 5-10 in this study is derived from memory B cells of patients recovered from the SARS-CoV-2 pneumonia. The profiles of affinity, in vitro blocking activity, and in vitro pseudovirus neutralizing activity have been confirmed in early stages. In this study, hACE2 transgenic mouse model were infected with SARS-CoV-2 to detect the prophylactic and therapeutic effects of Antibody 5-10 monoclonal antibody against SARS-CoV-2 infection on experimental animals.

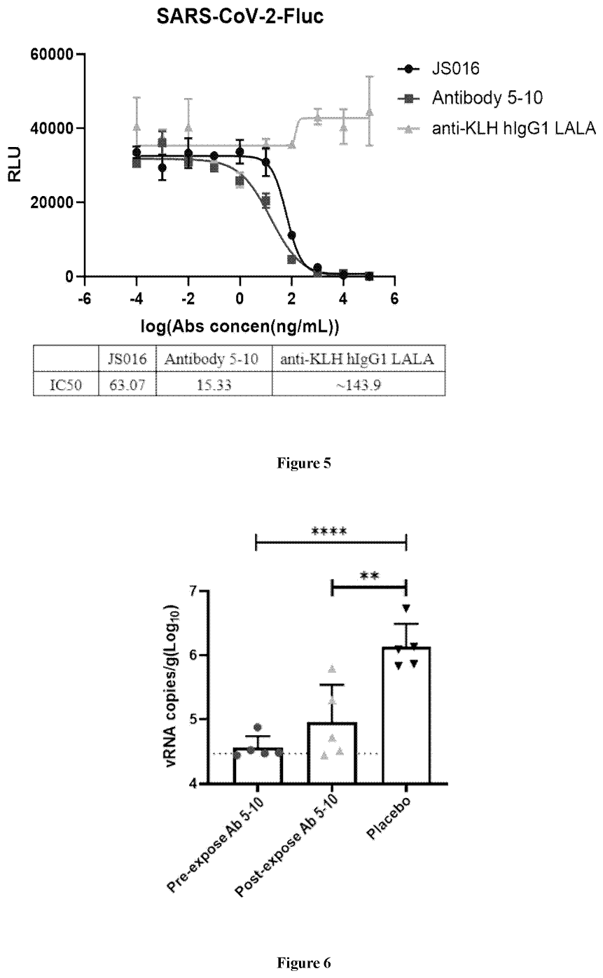

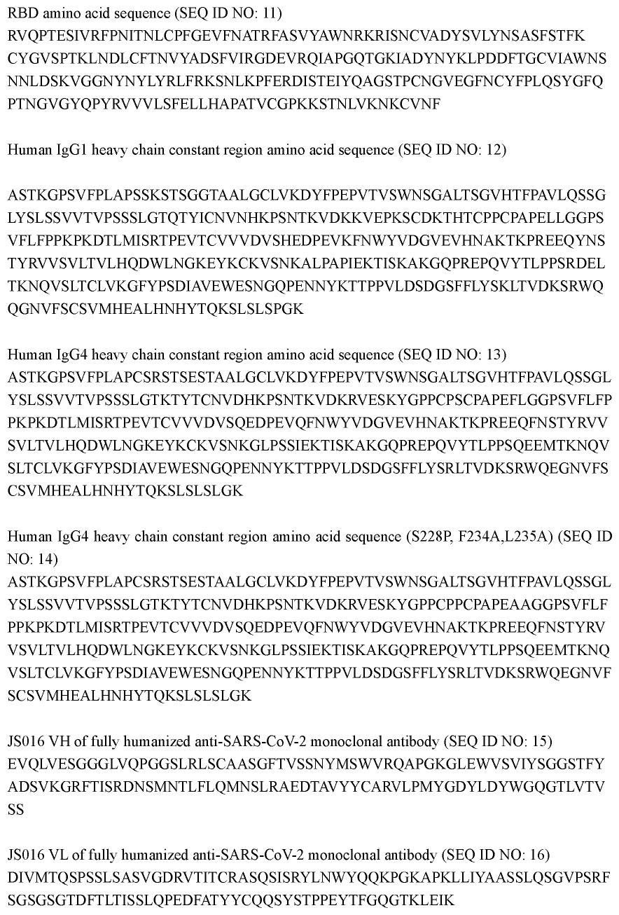

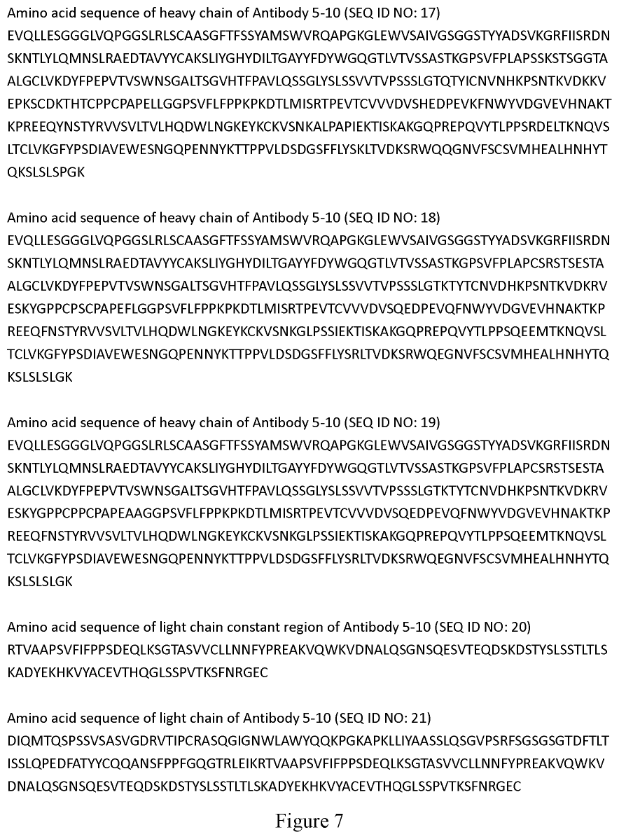

FIG. 6 showed that compared with the placebo group, the monoclonal antibody JS026 significantly reduced the viral load in the lungs of the mice, and exhibited great therapeutic effect. In addition, when Antibody 5-10 was used for prophylaxis, except for 1 of the 5 mice in this group with slight infection, the virus levels detected in the other 4 mice were close to the lower detection limit (dotted line) of the system, indicating that the antibody used for prophylaxis can be very effective in preventing or substantially decrease virus infection on mice. In this detection system, the monoclonal Antibody 5-10 used for prophylaxis or after the challenge can significantly reduce the viral load and pathological changes in lungs of mice. Antibody 5-10 proved to have great therapeutic and prophylactic effect against SARS-CoV-2 infection occurrence in experimental animals.

What is claimed is:

1. An isolated antibody or an antigen-binding fragment thereof capable of specifically binding to spike protein (e.g., S1) of SARS-CoV-2, comprising a heavy chain CDR 1 (HCDR1), HCDR2 and HCDR3 and/or a light chain CDR1 (LCDR1), LCDR2 and LCDR3, wherein: the HCDR1, the HCDR2, and the HCDR3 comprise amino acid sequences of SEQ ID NO: 1, SEQ ID NO: 2, and SEQ ID NO: 3, respectively, and the LCDR1, the LCDR2, and the LCDR3 comprise amino acid sequences of SEQ ID NO: 4, SEQ ID NO: 5, and SEQ ID NO: 6, respectively.

2. The antibody or an antigen-binding fragment thereof of claim 1, further comprising:
a) a heavy chain variable region (VH) comprising an amino acid sequence of SEQ ID NO: 7, or a sequence having at least 80% sequence identity thereof, and/or
b) a light chain variable region (VL) comprising an amino acid sequence of SEQ ID NO: 8, or a sequence having at least 80% sequence identity thereof.

3. The antibody or an antigen-binding fragment thereof of claim 1, wherein the antibody or antigen-binding fragment comprises: a VH comprising an amino acid sequence of SEQ ID NO: 7 or a sequence having at least 80% sequence identity thereof, and a VL comprising an amino acid sequence of SEQ ID NO: 8 or a sequence having at least 80% sequence identity thereof.

4. The antibody or antigen-binding fragment thereof of any of the preceding claims, which binds to receptor binding domain (RBD) of spike protein of SARS-CoV-2.

5. The antibody or antigen-binding fragment thereof of any of the preceding claims, further comprising one or more amino acid residue mutations yet retaining specific binding to RBD of spike protein of SARS-CoV-2.

6. The antibody or antigen-binding fragment thereof of any of the preceding claims, wherein at least one of the mutations is in one or more of the CDR sequences, and/or in one or more of the VH or VL sequences but not in any of the CDR sequences.

7. The antibody or antigen-binding fragment thereof of any of the preceding claims, further comprising an immunoglobulin constant region, optionally a constant region of human Ig, or optionally a constant region of human IgG.

8. The antibody or antigen-binding fragment thereof of any of the preceding claims, wherein the constant region comprises a constant region of human IgG1 or IgG4.

9. The antibody or antigen-binding fragment thereof of claim 9, wherein the heavy chain constant region of human IgG1 comprises SEQ ID NO: 12, or a sequence having at least 80% sequence identity thereof; or wherein the heavy chain constant region of human IgG4 comprises SEQ ID NO: 13, or a sequence having at least 80% sequence identity thereof.

10. The antibody or antigen-binding fragment thereof of claim 9, wherein the Fc region comprises one or more amino acid residue mutations conferring increased or reduced complement dependent cytotoxicity (CDC) or complement dependent cytotoxicity (ADCC) relative to wild-type constant region, or wherein the Fc region does not contribute to antibody dependent enhancement (ADE) of SARS-CoV-2 infection.

11. The antibody or antigen-binding fragment thereof of any of the preceding claims, further comprising:
a) a heavy chain comprising an amino acid sequence of SEQ ID NO: 17, 18, or 19; and/or
b) a light chain comprising an amino acid sequence of SEQ ID NO: 21.

12. The antibody or an antigen-binding fragment thereof of any of the preceding claims, which is fully human antibody, chimeric antibody, monoclonal antibody, a bispecific antibody, a multi-specific antibody, recombinant antibody, labeled antibody, bivalent antibody, anti-idiotypic antibody or a fusion protein.

13. The antibody or antigen-binding fragment thereof of any of the preceding claims, which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

14. The antibody or antigen-binding fragment thereof of any of the preceding claims, which is bispecific.

15. The antibody or antigen-binding fragment thereof of claim 14, capable of specifically binding to distinct epitopes on spike protein of SARS-CoV-2 or distinct antigens of SARS-CoV-2.

16. The antibody or antigen-binding fragment thereof of any of the preceding claims linked to one or more conjugate moieties.

17. An antibody or an antigen-binding fragment thereof, which competes for binding to RBD of spike protein of SARS-CoV-2 with the antibody or antigen-binding fragment thereof of any of claims 1-16.

18. A pharmaceutical composition comprising the antibody or antigen-binding fragment thereof of any of the preceding claims, and a pharmaceutically acceptable carrier.

19. The pharmaceutical composition of claim 18, comprising a combination of two or more of the antibodies or antigen-binding fragments thereof, wherein a second antibody of the combination bind to distinct epitopes on spike protein of the SARS-CoV-2, or specifically bind to SARS-CoV-2 in a non-competing manner.

20. The pharmaceutical composition of claim 18 or 19, further comprising an additional antibody capable of neutralizing SARS-CoV-2.

21. The pharmaceutical composition of claim 18 or 19, wherein the additional antibody binding to SARS-CoV-2 at an epitope or antigen distinct from that/those bound by the antibodies or antigen-binding fragments of any of claims 1-17.

22. The pharmaceutical composition of claim 21, wherein the additional antibody binding to non-RBD region of spike protein of the SARS-CoV-2.

23. The pharmaceutical composition of claim 18, wherein the composition comprises a cocktail of SARS-CoV-2 neutralizing antibodies that binds to at least two distinct epitopes on a SARS-CoV-2 serotype.

24. An isolated polynucleotide encoding the antibody or an antigen-binding fragment thereof of any of claims 1-17.

25. A vector comprising the isolated polynucleotide of claim 24, optionally the vector is an expression vector.

26. A host cell comprising the vector of claim 25.

27. A method of expressing the antibody or antigen-binding fragment thereof of any of claims 1-16, comprising culturing the host cell of claim 26 under the condition at which the vector of claim 26 is expressed.

28. A composition comprising a first mRNA polynucleotide encoding heavy chain or an antigen-binding fragment thereof of the antibody of any of claims 1-17, and a second mRNA polynucleotide encoding light chain or a fragment thereof of the antibody of any of claims 1-17.

29. The composition of claim 28, further comprises a pharmaceutically acceptable carrier.

30. A method of producing the antibody of any of claims 1-17, the method comprising administering the composition of claim 28 to a cell, wherein the first mRNA polynucleotide and the second mRNA polynucleotide are expressed in the cell, thereby producing the antibody.

31. A method of delivering the antibody of any of claims 1-17 in a subject, the method comprising:
administering the composition of claim 28 to a subject in need thereof, wherein the first mRNA polynucleotide and the second mRNA polynucleotide are expressed in the cell, thereby producing the antibody.

32. A method of treating or preventing SARS-CoV-2 infection in a subject, comprising administering to the subject an effective amount of the antibody or antigen-binding fragment thereof of any of claims 1-16, the pharmaceutical composition of any of claims 18-23, or the composition of any of claims 28-29.

33. The method of claim 32, wherein the subject is human or non-human animal.

34. The method of claim 32 or 33, wherein the subject has been identified as having SARS-CoV-2 infection, or is suspected of having SARS-CoV-2 infection, or is at risk of exposure to SARS-CoV-2.

35. The method of any of claims 32-34, wherein the administration is via oral, nasal, intravenous, subcutaneous, sublingual, or intramuscular administration.

36. The method of any of claims 32-35, further comprising administering an effective amount of a second therapeutic agent.

37. The method of any of claim 36, wherein the second therapeutic agent is selected from an antiviral agent such as a second SARS-CoV-2 neutralizing antibody, RNA dependent RNA polymerase inhibitor, a nucleoside analog, antiviral cytokines (such as interferons), or immunostimulatory agents.

38. A kit comprising an antibody of any of claims 1-16, and a second therapeutic agent.

39. A method of neutralizing SARS-CoV-2 in a subject, comprising administering the antibody, antigen-binding fragment thereof of any of claims 1-16 or the composition of any of claims 28-29 to the subject.

40. A method for preventing or reducing transmission of SARS-CoV-2 by a SARS-CoV-2 infected subject, comprising administering to the SARS-CoV-2 infected subject an effective amount of the antibody or antigen-binding fragment thereof of any of claims 1-16, and/or the pharmaceutical composition of any of claims 18-23, and/or the composition of any of claims 28-29.

41. A method of diagnosing SARS-CoV-2 infection in a subject, comprising: a) contacting a sample obtained from the subject with the antibody or antigen-binding fragment thereof of any of claims 1-16; b) determining presence or amount of SARS-CoV-2 in the sample; and c) correlating the presence or the amount of SARS-CoV-2 to existence or status of the SARS-CoV-2 infection in the subject.

42. A method of reducing viral load in a SARS-CoV-2 infected subject, comprising administering to the subject an effective amount of the antibody or antigen-binding fragment thereof of any of claims 1-16, and/or the pharmaceutical composition of any of claims 18-23, and/or the composition of any of claims 28-29.

43. Use of the antibody or antigen-binding fragment thereof of any of claims 1-16, and/or the composition of any of claims 28-29 in the manufacture of a medicament for treating or preventing SARS-CoV-2 infection in a subject; or for preventing, inhibiting progression of, and/or delaying the onset of SARS-CoV-2 infection or a SARS-CoV-2-associated condition in a subject; or for preventing or reducing transmission of SARS-CoV-2 by a SARS-CoV-2 infected subject; or for reducing viral load in a SARS-CoV-2 infected subject.

44. Use of the antibody or antigen-binding fragment thereof of any of claims 1-16, and/or the composition of any of claims 28-29 in the manufacture of a diagnostic reagent for diagnosing SARS-CoV-2 infection.

45. A kit comprising the antibody or antigen-binding fragment thereof of any of claims 1-16, useful in detecting SARS-CoV-2 presence.
